(19) United States
(12) Patent Application Publication
(10) Pub. No.: US 2005/0256131 A1
(43) Pub. Date: Nov. 17, 2005

(54) Pharmaceutical active substance combination and the use thereof

(76) Inventor: Carl-Fr Coester, Unna (DE)

Correspondence Address: MAINE & ASMUS, 100 MAIN STREET, P O BOX 3445, NASHUA, NH, 03061-3445, US

(21) Appl. No.: 10/515,984

(22) PCT Filed: May 21, 2003
(86) PCT No.: PCT/EP03/05300

§ 371 Date: Jul. 6, 2005

(30) Foreign Application Priority Data
May 24, 2002 (DE) 102 23 254.7
Nov. 25, 2002 (DE) 102 54 822.6
Mar. 7, 2003 (DE) 103 10 396.1

(51) Int. Cl. A61K 31/496

(52) U.S. Cl. 514/254.05

(57) ABSTRACT

The invention relates to the use of 2-[3-[4-(3-chlorophenyl)-1-piperazinyl]propyl]-5-ethyl-4-(2-phenoxyethyl)-2H-1,2,4-triazol-3(4H)-one (nefazodone) or a pharmaceutically compatible salt thereof for producing medicaments used in the treatment of Parkinson's disease.

The present invention relates to a pharmaceutical active substance combination comprising triazolinone derivatives and to the use of triazolinone derivatives and of the pharmaceutically acceptable salts thereof for the treatment of Parkinson's disease.

Parkinson's disease is associated with breakdown of nerve cells in the brain which are required to produce so-called dopamine. Dopamine is one of the messengers in the brain enabling information exchange between neighboring nerve cells. The decline in dopamine in parkinsonian patients means that other messengers predominate, in other words the equilibrium ratio of the messengers is disturbed. During the chronic disease, the messenger concentrations become grossly unbalanced. The symptoms of Parkinson's disease include in particular difficulties of coordination and impairments of mobility.

In most cases, the disease has its onset only at an advanced age, in particular between about the ages of 60 and 70. In rarer cases, however, the disease onset is also considerably earlier, in some circumstances even before the age of 30. It is estimated that more than one million patients in the world suffer from Parkinson's disease. One problem is that the disease is often not diagnosed in the early stage. Many patients are therefore not treated to begin with.

One of the conventional treatment methods is to replace the missing messenger dopamine by addition of appropriate medicaments. One problem with this is that on prolonged intake of L-dopa its activity declines so that the dosage must be increased. However, high L-dopa dosages lead to a number of side effects and, on prolonged intake, to undesired late sequelae.

This is a problem in particular for those patients in whom the disease becomes manifest at a comparatively young age. A high percentage of patients treated with L-dopa shows motor impairments after only a few years. For these reasons, there has recently been an increasing trend to employ, especially at the start of therapy, so-called dopamine agonists which are then in part combined with L-dopa in advanced stages of the disease.

Nefazodone, a phenoxyethyltriazolinone-phenylpiperazine, has become known as antidepressant in the art. It is assumed that the antidepressant effect of nefazodone is connected with the advancement of the serotonergic activity in the central nervous system. Nefazodone hydrochloride is ordinarily used as active substance in the corresponding medicaments. This agent is employed exclusively for depressive disorders. The manufacturer indicates some side effects in the information for use, it being indicated inter alia that relatively common side effects occurring to affect the nervous system are impairments of the coordination of movements (ataxia) and slowing of movements. The skilled worker concludes from this that nefazodone is contraindicated for the treatment of Parkinson's disease with which, after all, the aforementioned symptoms inter alia occur. The skilled worker thus had no reason to test the efficacy of nefazodone for the treatment of Parkinson's disease. The applicant is accordingly unaware of corresponding investigations in this direction.

The exact formula of nefazodone is represented for example in DE 34 43 820 C2 and corresponds to structural formula I indicated below

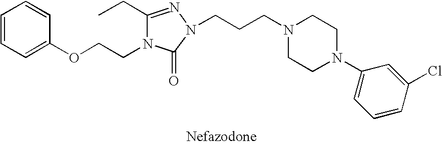

The exact name according to chemical nomenclature is 2-[3-[4-(3-chlorophenyl)-1-piperazinyl]propyl]-5-ethyl-4-(2-phenoxyethyl)-2H-1,2,4-triazol-3(4H)-one. In the aforementioned publication, nefazodone is referred to as agent having antidepressant activity. No further indications are mentioned.

Owing to the abovementioned fact, that the dopamine usage of parkinsonian patients increases after a prolonged treatment time, together with the increase in unwanted side effects and the occurrence of long-term damage, and in view of the wide distribution of the disease, whose incidence is moreover apparently increasing, there is a great national economic need to find medicaments which make possible a therapy in which the L-dopamine doses to be administered can be reduced.

The earlier, non-prior-published application DE 102 23 254.7, filed on May 24, 2002, of the applicant has described the use of the triazolinone derivatives 2-[3-[4-(3-chlorophenyl)-1-piperazinyl]propyl]-5-ethyl-4-(2-phenoxyethyl)-2H-1,2,4-triazol-3(4H)-one (nefazodone) or its pharmaceutically acceptable salts and the use of 2-[3-[4-(3-chlorophenyl)-1-piperazinyl]propyl]-1,2,4-triazolo[4,3-a]pyridin-3(2H)-one (trazodone) for the treatment of Parkinson's disease.

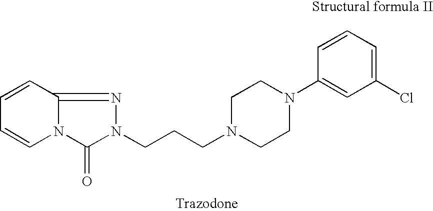

It was surprisingly possible to establish that these substances, which have previously been known only as antidepressants, show an exceptionally good therapeutic effect in the treatment of the pathological symptoms of Parkinson's disease. Following these findings which are of medical and national economic importance, further clinical studies were carried out with series of tests on patients, through which it was possible to confirm the astonishing efficacy of said active substances. However, in these studies initially only either nefazodone or trazodone were employed in each case as active substance on its own for the treatment of patients. A disadvantage which has emerged for the active substance trazodone is that it causes a certain tiredness in the patients after intake. Intake of this active substance by the patients during the day is therefore inadvisable.

In the treatment of parkinsonian patients with the dopamine medicaments customary to date, it has to date been usual on intake of the dopamine composition in the evening additionally to add a so-called depot composition to the medicament comprising the active substance dopamine, because otherwise the dopamine was released too rapidly, which is unwanted, in the metabolism of the sleeping patient, so that the effect of the dopamine disadvantageously did not persist over a sufficiently long time until the next morning. The depot compositions normally used in this case are very costly in the prior art.

The object of the present invention is accordingly to provide a composition which reduces the dopamine usage of parkinsonian patients, displays good efficacy for the treatment of the pathological symptoms and moreover shows minimal or only insignificant side effects.

Achievement of this object provides an inventive use as claimed in any of claims 1, 2 or 3, and a pharmaceutical active substance combination as claimed in claim 17.

Investigations for the purposes of the present invention have shown that intake of the aforementioned inventive substances by parkinsonian patients surprisingly leads to a considerable reduction in the dopamine usage. It was possible to show in some patients that parkinsonian patients regularly taking L-dopamine are able to reduce very considerably their dopamine usage compared with formerly on simultaneous intake of nefazodone. The dopamine dose necessary on additional intake of nefazodone can in many cases be reduced for example to one half or even one third of the dose previously necessary on treatment with dopamine alone. Moreover, according to the invention there is not only a reduction in the daily dose of dopamine, but remarkably the intake of nefazodone also improves the time distribution of the dopamine which has been taken in the patient's body. Dopamine usually has a relatively short half-life in the human body, so that its effect does not persist for very long with pure dopamine therapy therefore, the parkinsonian patient must take the dopamine distributed relatively frequently over the day, for example at an interval of two to two and a half hours. Since the effect of dopamine is additionally impaired by simultaneous intake of food, it is recommended that no food be consumed for a period after its intake. This leads to a considerable impairment of the quality of life of parkinsonian patients, especially when the disease is already in an advanced stage and therefore high dosages of dopamine and relatively frequent intake at short intervals of time is necessary. The inventive use in particular of nefazodone or derivatives thereof in patients who take dopamine at the same time by contrast advantageously leads to the effect of dopamine being distributed in time. Evidently, for a reason which is as yet unknown, a depot effect arises, with which the dopamine in the patient's body is released more slowly owing to the intake of nefazodone. It is thus possible for dopamine intake to take place not only in lower doses but also at larger intervals in time.

It was additionally established that the inventive use of, in particular, nefazodone and derivatives thereof leads to a reduction in the side effects associated with conventional Parkinson's medicaments comprising dopamine. For example, one unpleasant side effect of dopamine, namely the occurrence of uncontrolled motor activity in the patient, is positively influenced by nefazodone.

A further advantage of the inventive use of nefazodone is that it can easily be administered orally, especially in tablet form, in contrast to other anti-Parkinson's medicaments which have been disclosed recently and which have to be injected and, in some cases, can be injected only by the physician if the syringe must be placed for example in the head region in the direct vicinity of the brain region.

If the active substance of the invention is administered in tablet form, such a table normally comprises conventional excipients besides the active substance itself. Examples of suitable excipients are those used for the commercially available medicament “Nefadar [symbol] ”, and these are in particular microcrystalline cellulose, povidone, poly(O-carboxymethyl)starch, sodium salt, colloidal silicon dioxide, magnesium stearate, iron oxide or the like.

In addition, nefazodone or the usual medicament-compatible salts thereof is generally well tolerated and shows only relatively few or rare serious side effects.

A preferred dosage for the purposes of the present invention comprises daily intake of a few 100 mg, this intake preferably being distributed over the day in a plurality of doses, preferably through intake of tablets. The tablets normally comprise the active substance in an amount of 100 mg or 200 mg. A preferred daily dosage is, for example, in the range from about 300 to 600 mg in a day, so that this can be administered by intake two to three times a day in single doses of 100 mg or 200 mg. For example, if a total dose of 500 mg a day is intended, it is possible to take 200 mg in the morning, 200 mg at midday and 100 mg in the evening. The inventive administration of the nefazodone products led to it being possible considerably to reduce the dopamine usage of the patient. For example, it was possible to reduce the daily dose necessary for a patient whose disease was already in an advanced stage from the 900 mg to 1000 mg of dopamine a day before the inventive treatment with nefazodone to a total daily dose of only 300 to 400 mg, in other words to about one third. Intake of L-dopamine was possible in considerably smaller single doses and simultaneously with a greater interval in time, for example in three single doses of about 125 mg, which were taken for example three times a day, specifically in the morning, at midday and in the evening. This had the considerable advantage for the patient that, because of the larger interval in time of L-dopamine intake, it was possible to take meals undisturbed in a usual rhythm as for a healthy person.

Besides nefazodone, an active substance suitable for the inventive use is in particular another triazolone which likewise comprises as substituent a phenylpiperazine group which is linked via a propyl group to a nitrogen atom of the triazolone ring. It is the 1,2,4-triazolo-[4,3-a]pyridine which has become known under the name trazodone and is described in U.S. Pat. No. 4,338,317 as antidepressant. The structural formula II of trazodone is represented below.

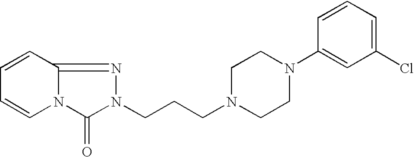

The exact name according to chemical nomenclature is 2-[3-[4-(3-chlorophenyl)-1-piperazinyl]propyl]-1,2,4-triazolo[4,3-a]pyridin-3(2H)-one.

It is to be assumed that the triazolinone group which is present in both trazodone and in the abovementioned nefazodone, and the substituents which exhibit a plurality of agreements in both cases, namely the propylphenylpiperazinyl group on the one hand and the substituents in position 4 and 5 of the triazole ring on the other hand, are responsible for the inventive effect on Parkinson's disease. The exact mechanism of action has not yet been investigated.

Investigations for the purposes of the present invention have shown that additional intake of caffeine, for example in tablet form, assists the mentioned positive effect of nefazodone and can lead to a further reduction in the dopamine usage of the patient. It is recommended in this connection for example to take a single dose of about 50 mg to about 0.2 g of caffeine in tablet form. Caffeine tablets with this active substance dose are commercially available. It has additionally been established that intake of acetylsalicylic acids can also assist the mentioned positive effects of nefazodone, so that supplementary therapy with acetylsalicylic acid may also be advisable. An appropriate example is intake of acetylsalicylic acid in tablet form with single doses of, for example, 500 mg per tablet. Combinations of caffeine and acetylsalicylic acid active substances in one tablet are also possible, resulting in the advantage that the patient has to take only one medicament.

In the further investigations described previously, it was surprisingly possible to establish that said fact that the active substance trazodone causes a certain tiredness in the patient can be utilized therapeutically in a particularly advantageous way. The medicament with the active substance trazodone was administered to the patients in the evening. The active substance trazodone had a sleep-promoting effect on the patient. It is assumed that on treatment of parkinsonian patients with nefazodone or else trazodone it is still worthwhile for the patient to take dopamine as supplement. This will certainly also depend on the particular patient and the stage of the disease. The active substances nefazodone and trazodone are therefore at least able to achieve a reduction in the necessary dose of dopamine to be taken. However, to date, for the aforementioned reasons, the patients have been given the dopamine to be administered in the evening in conjunction with a depot composition.

It was then possible to establish by further investigations that the active substance trazodone itself causes a depot effect with the dopamine medicament which is additionally taken. This finding leads to the crucial therapeutic advantage that it is now possible on intake of trazodone in the evening to take the dopamine medicament which is to be administered in addition, without the depot composition which was necessary to date, there being nevertheless the favorable slow release of the dopamine during the period of sleep. Since the depot composition is dispensed with, this leads to a considerable cost advantage of the therapy. Since, in addition trazodone has a sleep-promoting effect, the dopamine usage in the period of sleep is also reduced moreover. The patient is then able the next morning to take the nefazodone which is more suited to administration during the day. It emerged from the investigations carried out that, after this nefazodone intake, the patients who had taken trazodone the previous evening gave a relaxed impression. Thus, combination of the active substances nefazodone and trazodone in the therapy of Parkinson's disease leads to remarkably good results. Moreover, combined therapy with the two active substances, which can be taken simultaneously, separately or sequentially, have advantages in several respects which go beyond the effect on therapy with in each case only one of the two individual active substances.

Sequential use of the two active substances mentioned in the therapy of Parkinson's disease is particularly advantageous and preferred. It is very particularly advantageous to use trazodone or a pharmaceutically acceptable salt thereof or a composition containing the latter in the evening or before retiring to sleep in combination with the use of nefazodone or of a pharmaceutically acceptable salt thereof or of a composition containing the latter during the day in one or more single doses.

It is particularly preferred to use the mentioned active substances nefazodone and/or trazodone in medicaments which are in tablet form. It is regarded as advantageous in this connection for a tablet intended for a single dose to contain between about 50 mg and about 200 mg of one of the active substances nefazodone or trazodone or in each case a corresponding dose of the two active substances. It may moreover be advantageous to combine the two active substances in one tablet, in which case the single dose of the individual active substance in the tablet can be reduced, so that for example a tablet could also contain in each case only 25 mg of nefazodone and 25 mg of trazodone. It is additionally possible for the respective ratio of the contents of active substances in individual tablets or combination products which are to be taken simultaneously or sequentially to be varied, it being appropriate for the aforementioned reasons to choose a higher dosage of the active substance nefazodone for intake during the day than the respective content of trazodone, and to shift this ratio towards the evening so that tablets to be taken in the evening preferably comprise mainly the active substance trazodone and only a smaller amount of the active substance nefazodone. It is, of course, also possible to have the patient take one or more tablets comprising only trazodone in the evening.

The active substance trazodone may be present in medicaments comprising the latter as pharmaceutically acceptable salt, for example in the form of a hydrochloride.

Parkinsonian patients frequently suffer from depression because of their disease. Owing to the fact that intake of nefazodone, but also trazodone, by depressive patients not suffering from Parkinson's led to side effects affecting the nervous system, for example impairments of the coordination of movements (ataxia) and slowing of movements, it has to date been assumed that nefazodone or trazodone is contraindicated for patients suffering from depression but not affected by Parkinson's disease. The investigations prompted by the applicant after the establishment of the efficacy of the active substances nefazodone and trazodone in the treatment of the symptoms of Parkinson's disease have led to the realization that the antidepressant effect of the active substances nefazodone and trazodone is particularly evident in parkinsonian patients, especially those treated in parallel with dopamine-containing medicaments. It is therefore, according to the new realizations emerging from the investigations in connection with the present invention, particularly advisable to employ the active substances nefazodone and trazodone each on its own, in combination with one another and/or in combination with dopamine-containing medicaments specifically also for the treatment of depression in parkinsonian patients.

Further studies on patients for the purposes of the present invention have revealed that although a good effect in relation to a regression of the symptoms was observed in some patients treated with nefazodone or trazodone for Parkinson's disease, it was possible to observe an excellent effect in a few patients. Since there was no explanation of this difference in the response to the active substances nefazodone and trazodone, this prompted further investigations to be carried out in order to elucidate the relationships.

It was possible to establish, surprisingly, in the further studies mentioned, that intake of an antihistamine shows a good effect in the treatment of the symptoms of Parkinson's disease. A particularly good effect is shown by the antihistamine with the active substance cetirizine or a pharmaceutically acceptable salt thereof, in particular cetirizine dihydrochloride. This is a relatively widely used antihistamine which is employed for the treatment of allergies such as, for example, hay fever, pruritic symptoms and the like. Cetirizine has the advantage that no relevant side effects are known. The medicament is very well tolerated and can even be taken by children. It acts relatively rapidly, and no noteworthy interactions with other medicaments are known. A single daily intake usually suffices. The active substance cetirizine can be taken for example in tablet form or, for example, also in the form of a solution or suspension which is administered orally for example as liquid. The medicament can usually be obtained without prescription. The active substance cetirizine acts by blocking the histamine in the body.

According to statements in the literature, cetirizine is an antihistamine with a predominantly peripheral activity. Compared with other antihistamines, it is said to have only a slight central sedative effect. The chemical structural formula of cetirizine dihydrochloride is evident from the following depiction of

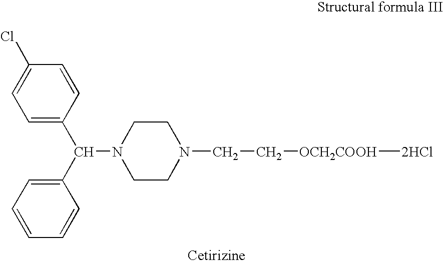

Despite the relatively low central sedative effect, according to statements in the literature a reduction in psychomotor performance is found on administration of cetirizine to patients. This means that intake of the active substance by parkinsonian patients would be contraindicated according to the prior art. It is all the more surprising to find according to the invention that extremely good results can be achieved in the therapy of Parkinson's disease in particular on combination of cetirizine with nefazodone and/or trazodone.

It is particularly preferred for the purposes of the present invention to use the active substance cetirizine or one of its pharmaceutically acceptable salts in conjunction with one of the triazolone derivatives mentioned at the outset, nefazodone and/or trazodone, for the treatment of Parkinson's disease. The present invention thus likewise relates to an active substance combination of cetirizine and trazodone or cetirizine and nefazodone, it being possible to employ this active substance combination as combination product for simultaneous, separate or sequential use in the therapy of Parkinson's disease. This means that an active substance combination of cetirizine and nefazodone or cetirizine and trazodone may be present in a medicament, the two active substances may be present in separate medicaments which are taken simultaneously, or the two active substances are present in separate medicaments which are taken at different times, but in a combined therapy. Intake of cetirizine can take place for example once a day, because experience shows that the effect of a tablet persists for 24 hours. The active substance nefazodone or trazodone which is additionally to be taken can likewise be in tablet form as pure active substance or as pharmaceutically acceptable salt, for example in the form of the hydrochloride. The active substance nefazodone and/or trazodone can be taken a plurality of times a day, depending on the severity of the disease, a usual single dose on intake of tablets ordinarily being between about 50 mg and about 200 mg of one of the active substances. It is also possible for the two active substances nefazodone and trazodone to be combined in one medicament. In this case, the active substance dose of the individual active substance can be reduced correspondingly.

In cases where the parkinsonian patient additionally suffers from allergies, the combination of nefazodone and/or trazodone with the antihistamine cetirizine is particularly advantageous because the symptoms of the allergy are treated at the same time. However, since no relevant side effects are known for cetirizine, this active substance can unhesitatingly also be taken over a prolonged period by parkinsonian patients not suffering from allergies.

The inventive combination therapy composed of nefazodone and/or trazodone and the antihistamine cetirizine has the advantage that a considerably lower dose of dopamine, whose intake is also necessary where appropriate, is possible than without the intake of the aforementioned active substances of the invention. The interaction of the antihistamine cetirizine with said triazolinone derivatives, especially with nefazodone or trazodone, is particularly surprising because an interaction between the antihistamine and other Parkinson's medicaments on the market has not been found. The interaction which evidently exists between the antihistamine and the triazolinone derivative cannot as yet be scientifically explained because of the complexity of the biochemical and physiological processes. However, the applicant has been able to establish that the antihistamine enhances the effect of the nefazodone or trazodone in the treatment of the symptoms of Parkinson's disease.

1. The use of 2-[3-[4-(3-chlorophenyl)-1-piperazin-yl]propyl]-5-ethyl-4-(2-phenoxyethyl)-2H-1,2,4-triazol-3 (4H)-one (nefazodone) or of a pharmaceutically acceptable salt thereof for producing medicaments for the treatment of Parkinson's disease.

2. The use of 2-[3-[4-(3 -chlorophenyl)-1 -piperazin-yl]propyl]-1,2,4-triazolo[4,3-a]pyridin-3(2H)-one (trazodone) or of a pharmaceutically acceptable salt thereof and of 2-[3-[4-(3-chlorophenyl)-1-piperazinyl]propyl]-5-ethyl-4-(2-phenoxyethyl)-2H-1,2,4-triazol-3(4H)-one (nefazodone) or of a pharmaceutically acceptable salt thereof for producing medicaments for the treatment of Parkinson's disease.

3. The use of cetirizine or of a pharmaceutically acceptable salt thereof for producing medicaments for the treatment of Parkinson's disease.

4. The use as claimed in claim 3, characterized in that the latter includes cetirizine dihydrochloride.

5. The use as claimed in claim 3, characterized in that the medicament includes a single dose of cetirizine of at least about 5 mg, preferably at least about 10 mg.

6. The use as claimed in claim 3, characterized in that the cetirizine is in tablet form or in the form of a solution or suspension.

7. The use as claimed in claim 2, in which the composition comprising the trazodone or a pharmaceutically acceptable salt thereof is intended for intake in the evening.

8. The use as claimed in claim 2, in which the medicament is in tablet form, where the tablet intended for a single dose comprises between about 50 mg and about 200 mg of one of the active substances nefazodone or trazodone or in each case a corresponding dose of both active substances.

9. The use as claimed in claim 2 for producing medicaments for the treatment of depression in parkinsonian patients.

10. The use as claimed in claim 1 in patients simultaneously treated with L-dopamine.

11. The use as claimed in claim 1, characterized in that a daily dose totaling between about 100 mg and about 800 mg, where appropriate in a plurality of single doses, is administered to the patient.

12. The use as claimed in claim 1, characterized in that a daily dose of between about 300 and about 600 mg is administered to the patient.

13. The use as claimed in claim 1, characterized in that the nefazodone or its pharmaceutically acceptable salt is administered in two to three single doses.

14. The use as claimed in claim 1, characterized in that the nefazodone or its pharmaceutically acceptable salt is administered in one or more single doses each of about 100 mg to about 200 mg.

15. The use as claimed in claim 1, characterized in that the nefazodone or its pharmaceutically acceptable salt is administered in tablet form intended for oral intake.

16. The use as claimed in claim 1, in conjunction with intake of a caffeine- and/or acetylsalicylic acid-containing composition in the same period for the treatment of Parkinson's disease.

17. A pharmaceutical active substance combination comprising 2-[3-[4-(3-chlorophenyl)-1-piperazinyl]propyl]-5-ethyl-4-(2-phenoxyethyl)-2H-1,2,4-triazol-3 (4H)-one (nefazodone) or a pharmaceutically acceptable salt thereof and 2-[3-[4-(3-chlorophenyl)-1-piperazinyl]propyl]-1,2,4-triazolo[3,4-a]pyridin-3(2H)-one (trazodone) as combination product for simultaneous, separate or sequential use in the therapy of Parkinson's disease.

18. The pharmaceutical active substance combination as claimed in claim 17 comprising at least one antihistamine or a pharmaceutically acceptable salt thereof besides nefazodone or trazodone as combination product for simultaneous, separate or sequential use in the therapy of Parkinson's disease.

19. The pharmaceutical active substance combination as claimed in claim 18, characterized in that the latter includes cetirizine or one of its pharmaceutically acceptable salts as antihistamine.

20. The pharmaceutical active substance combination as claimed in claim 17, characterized in that at least the nefazodone/trazodone is in tablet form, where a tablet intended for a single dose comprises between about 50 mg and about 200 mg of at least one of the active substances nefazodone and/or trazodone or in each case a corresponding dose of both active substances.

21. The pharmaceutical active substance combination as claimed in claim 18, characterized in that the antihistamine includes cetirizine or its hydrochloride.

22. The pharmaceutical active substance combination as claimed in claim 21, characterized in that cetirizine or one of its pharmaceutically acceptable salts is in tablet form, and a single dose comprises at least about 5 mg, preferably at least about 10 mg, of this active substance.

23. The use as claimed in any of claim 2 in patients simultaneously treated with L-dopamine.

24. The use as claimed in any of claim 3 in patients simultaneously treated with L-dopamine.

25. The use as claimed in claim 2, characterized in that a daily dose totaling between about 100 mg and about 800 mg, where appropriate in a plurality of single doses, is administered to the patient.

26. The use as claimed in any of claim 2, characterized in that a daily dose of between about 300 and about 600 mg is administered to the patient.

27. The use as claimed in any of claim 2, characterized in that the nefazodone or its pharmaceutically acceptable salt is administered in two to three single doses.

28. The use as claimed in any of claims 2, characterized in that the nefazodone or its pharmaceutically acceptable salt is administered in one or more single doses each of about 100 mg to about 200 mg.

29. The use as claimed in any of claims 2, characterized in that the nefazodone or its pharmaceutically acceptable salt is administered in tablet form intended for oral intake.

30. The use as claimed in any of claims 2 in conjunction with intake of a caffeine- and/or acetylsalicylic acid-containing composition in the same period for the treatment of Parkinson's disease.

31. The use as claimed in any of claims 3 in conjunction with intake of a caffeine- and/or acetylsalicylic acid-containing composition in the same period for the treatment of Parkinson's disease.
